(19) United States
(12) Patent Application Publication
(10) Pub. No.: US 2003/0049805 A1
(43) Pub. Date: Mar. 13, 2003

(54) Fermentative production of L-lysine

(75) Inventors: Kazuo Nagase, Kawasaki-shi (JP); Hisashi Yasueda, Kawasaki-shi (JP); Shinichi Sugimoto, Kawasaki-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: AJINOMOTO CO. INC, Tokyo (JP)

(21) Appl. No.: 10/274,883

(22) Filed: Oct. 22, 2002

Related U.S. Application Data
(63) Continuation of application No. 09/694,033, filed on Oct. 23, 2000.

(30) Foreign Application Priority Data
Oct. 22, 1999 (JP) 11-300500

(51) Int. Cl. C12P 13/08; C07H 21/04; C12P 21/02; C12N 1/21; C12N 15/74; C12N 9/12

(52) U.S. Cl. 435/115; 435/69.1; 435/194; 435/252.3; 435/320.1; 536/23.2

(57) ABSTRACT

The present invention provides Methylobacillus bacteria containing an aspartokinase gene and/or a dihydrodipicolinate synthase gene wherein the activities of these genes are insensitive to L-lysine feedback inhibition and methods of producing L-lysine by culturing the Methylobacillus bacteria.

### FIELD OF THE INVENTION

This invention relates to a method of producing L-lysine by fermentation and microorganisms capable of such production.

### BACKGROUND OF THE ART

Producing L-lysine by fermentation typically involves culturing bacterial strains isolated from nature. In addition, bacterial mutants have been used to improve L-lysine productivity. Many previous bacterial mutants that produce L-lysine are aminoethylcysteine (AEC) resistant strains and are either Brevibacterium, Corynebacterium, Bacillus, or Escherichia bacterium.

Transformation of bacteria with recombinant DNA to enhance L-lysine production has been described in U.S. Pat. No. 4,278,765. For example, transformation of Escherichia bacteria with a wild type dihydrodipicolinate synthase gene (abbreviated as “DDPS” hereinafter) and the resultant enhanced fermentative production of L-lysine is disclosed in U.S. Pat. No. 4,346,170 and Applied Microbiology and Biotechnology, 15, p227-231 (1982). However, the DDPS gene used was a wild type gene and as such was subject to L-lysine feedback inhibition thereby limiting the production of L-lysine. To render the bacterium desensitized from L-lysine feedback inhibition, aspartokinase (abbreviated as “AK” hereinafter) along with DDPS has been amplified in the bacteria (WO95/16042). The resultant bacteria is insensitive to L-lysine feedback inhibition and thus L-lysine production was greatly improved.

Dihydrodipicolinate synthase is an enzyme that synthesizes dihydrodipicolinate through dehydration condensation of aspartate-beta-semialdehyde and pyruvic acid. This reaction serves as an entry point into the L-lysine biosynthesis pathway within the aspartic acid amino acid pathway. In Escherichia bacteria, both AK and DDPS are known to be involved in important rate-limiting steps in L-lysine production. The DDPS is encoded by the dapA gene in Escherichia coli which has been cloned and the nucleotide sequence determined (Richaudm F. et al. J. Bacteriol., 297 (1986)).

AK is an enzyme that catalyzes the production of aspartate-beta-semialdehyde from aspartic acid and is sensitive to feedback inhibition. E. coli has three isozymes of AK (AKI, AKII and AKIII). Two of them are complex enzymes that also have homoserine dehydrogenase activity (abbreviated as “HD” hereinafter). In contrast, AKIII has only a single enzymatic activity, is encoded on the lysC gene and is known to be sensitive to feedback inhibition and repression by L-lysine.

Methods of fermentative amino acid production using methanol, a carbon source that is inexpensive and easy to obtain in large quantities, have been described. Examples of such production methods have been described for different microorganisms: Achromobacter or Pseudomonas (Japanese Patent Publication No. 25273/1970), Protaminobacter (Japanese Patent Application laid-open No. 125590/1974) Protaminobacter or Methanomonas (Japanese Patent Application laid-open No. 25790/1975), Micocyclus (Japanese Patent Application laid-open 18886/1977), Methylobacillus (Japanese Patent Application laid-open 91793/1992) and Bacillus (Japanese Patent Application laid-open 505284/1991 European Patent Application No. 90906690.4).

Methods of screening Methylobacillus bacteria for mutants resistant to amino acid feedback inhibition or metabolic inhibitors of amino acid are known (Japanese Patent Application laid-open 91793/1992). Additionally, screening mutants resistant to halogenated pyruvic acid has been disclosed (Japanese Patent Application laid-open 133788/1994). However, nothing is known whether the method of feedback insensitive DDPS or AK in Methylobacillus is effective in the production of L-lysine. Especially, nothing is known as for method using recombinant DNA technique and introducing genes into Methylobacillus that enable lysine biosynthesis.

### SUMMARY OF THE INVENTION

The present invention is accomplished in view of the aforementioned technical aspect, and its object is to provide improved fermentative methods of producing L-lysine using methanol as a major carbon source.

Accordingly, one object of the present invention is a Methylobacillus bacterium having one or both of an AK gene and a DDPS gene wherein both genes are insensitive to L-lysine feedback inhibition.

Another object of the invention is where the AK and the DDPS gene are obtained from Escherichia coli or where the DDPS gene is obtained from a Corynebacterium.

In the production of L-lysine an object of the present invention is a method for producing L-lysine which comprises culturing the Methylobacillus bacteria containing the AK and DDPS genes in a media containing methanol and subsequently collecting the L-lysine produced.

The inventors of present invention assiduously studied in order to achieve aforementioned objects. As a result, they successfully constructed Methylobacillus carrying AK and DDPS whereby the microorganisms are free from L-lysine feedback inhibition. This constructed Methylbacillus has been found to yield considerable quantities of L-lysine when cultivated in a suitable medium.

### BRIEF DESCRIPTION OF THE DRAWINGS

FIG. 1 is a schematic overview of the construction of the pRSFTC plasmid from pVIC40 and pBR322.

FIG. 2 is a schematic overview of the construction of the pRSFT806 from pRSFTC and pRSFD80.

### PREFERRED EMBODIMENTS OF THE INVENTION

AK and DDPS DNA can be obtained from any microorganism that carries AK and/or DDPS which are insensitive to L-lysine feedback inhibition in Methylobacillus. The bacterium may be a wild-type strain or a mutant strain. The genes may be isolated from the same microorganism or be isolated from two different microorganisms. Preferred mutants are those derived from Escherichia coli K-12, from Methylobacillus glycogenes NCIMB11375 Corynebacterium glutamicum ATCC138 69 and mutant derived from Corynebacterium glutamicum. More preferably, one can obtain feedback-insensitive AK and DDPS gene from Escherichia coli by the method disclosed in the W095/16042. The DDPS gene isolated from Corynebacterium glutamicum, is known not to be sensitive to feedback inhibition even in the wild type strain (Japanese Patent Application laid-open 62866/1994) and as such, this Corynebacterium is suitable for obtaining DNA.

Any plasmid vector containing feedback-insensitive AK and DDPS genes can be used as long as it can be introduced and expressed in a Methylobacillus bacterium. Examples of such vectors are pMF42 (Gene, 44, 53 (1990)), pRP301, and pTB70 (Nature, 287, 396, (1980)).

Any method of transformation can be used to introduce plasmid vectors into Methylobacillus, as long as it results in good transformation efficiency. Examples of such transformation methods involve introduction of plasmid DNA into Methylobacillus from a E. coli S17-1 strain by conjugation whereby the two bacteria are cultured on the same agar media plate as disclosed in Methods in Enzymology, 118, 640 (1986). Direct introduction of plasmid by electroporation can also be used (Canadian Journal of Microbiology, 43, 197 (1997)).

Alternatively, rather than introduction of the DNA using plasmid DNA, the AK and DDPS genes may be integrated into the bacterial chromosome or the AK and/or DDPS genes can be modified or mutagenized into feedback insensitive genes directly in the chromosome.

According to the present invention, any Methylobacillus strain may be used. Examples of such strains include Methylobacillus glycogenes NCIMB11375 and Methylobacillus flagelatum ATCC51484. The Methylobacillus strains carrying a plasmid(s) containing the AK and DDPS genes in methanol media produce considerable amounts of L-lysine in the culture media after culturing.

The microorganism used in the present invention can be cultivated by the ordinary method for methanol utilizing bacteria. Either nutrient or synthetic medium can be used as far as it contains carbon source, nitrogen source, inorganic ions and other desired trace organic compounds.

In culturing the Methylobacillus, methanol is used as a major carbon source. Preferably, methanol is in the media in an amount from 0.01 to 30% w/v. This concentration includes 0.05, 0.1, 0.4, 0.7, 1.0, 5.0, 7.5, 10, 13, 16. 19, 22, 25, 27% w/v and all values and subranges therebetween. As for a source of nitrogen, ammonium sulfate and other known nitrogen sources can be used in the medium. In addition, potassium phosphate, sodium phosphate, manganese sulfate, ferrous sulfate and manganese sulfate may be added into the medium as is known in the art.

Culturing of the Methylobacillus is performed under aerobic conditions by shaking or aeration plus agitation, at a pH of from 6 to 8, and at a temperature of from 25° C. to 37° C. The culture typically continues for 24 to 120 hours. After culturing the L-lysine can be recovered from the culture medium. The L-lysine can be further purified by precipitation and/or chromatography, e.g., ion-exchange chromatography.

Having generally described this invention, a further understanding can be obtained by reference to certain specific examples which are provided herein for purposes of illustration only, and are not intended to be limiting unless otherwise specified.

### EXAMPLES

The media used in the examples are:

| L medium | | |
|---|---|---|
| Bacto trypton(Difco) | 1% | |
| Yeast Extract(Difco) | 0.5% | |
| NaCl | 0.5% | |
| L agar medium | | |
| L medium | | |
| Bacto agar(Difco) | 1.5% | |
| PY medium | | |
| Nutrient Broth (Difco) | 1.0% | |
| Yeast extract (Difco) | 0.25% | |
| Methanol | 10.0 | ml/l(pH 7.0) |
| PY agar medium | | |
| PY medium | | |
| Bato agar (Difco) | 1.5% | |
| M1 medium | | |
| (NH₄)₂SO₄ | 0.2% | |
| K₂HPO₄ | 0.7% | |
| KH₂PO₄ | 0.1% | |
| MgSO₄ | 0.05% | |
| NaCl | 0.01% | |
| FeSO₄ | 10.0 | mg/l |
| MnSO₄ · 5H₂O | 8.0 | mg/l |
| Vitamin B1 | 1.0 | mg/l |
| Biotin | 10.0 | ug/l |
| Methanol | 5.0 | ml/l(pH 7-0) |
| M1 agar medium | | |
| M1 medium | | |
| Bacto agar (Difco) | 1.5% | |

The pH of all of the media was adjusted using NaOH or HCl. Media were steam-sterilized at 120° C. for 15 min. If the media contained methanol, the media were prepared without adding methanol and following steam sterilization, methanol which was filtered through a Membrane filter 0.45 um (Milipore) was added to the sterilized media.

The plasmid RSFD80 was constructed as described in W095/16042. This plasmid contains a feedback-insensitive DDPS gene, a feedback-insensitive AK gene and a replication origin derived from broad host range plasmid. E. coli JM109 which harbors RSFD80 was named AJ12396 and was deposited at National Institute of Bioscience and Human-Technology, Agency of Industrial Science and Technology, Ministry of International Trade and Industry (postal code 305-8566, 1-3 Higashi 1-chome, Tsukuba-shi, Ibaraki-ken, Japan) under the accession number FERM BP-4859. AJ12396 was cultured in 50 ml of L medium containing 20 mg/l of streptomycin overnight at 37° C. and the RSFD80 plasmid was purified using Wizard Plus Midipreps DNA Purification System (Promega).

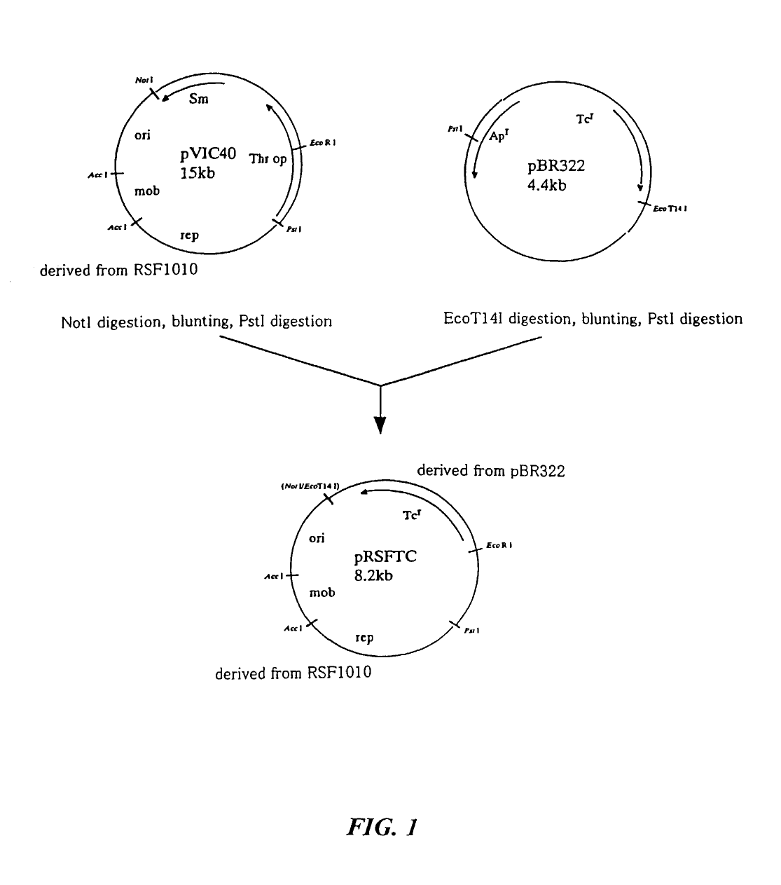

The plasmid pRSFTC was constructed from pVIC40 and pBR322 according to the method disclosed in W095/16042 (see FIG. 1). JM109 was transformed with the pRSFTC plasmid and the resultant transformant was cultured in 50 ml of L medium containing 10 mg/l of tetracyclin overnight at 37° C. and pRSFTC was purified using the Wizard Plus Midipreps DNA Purification System.

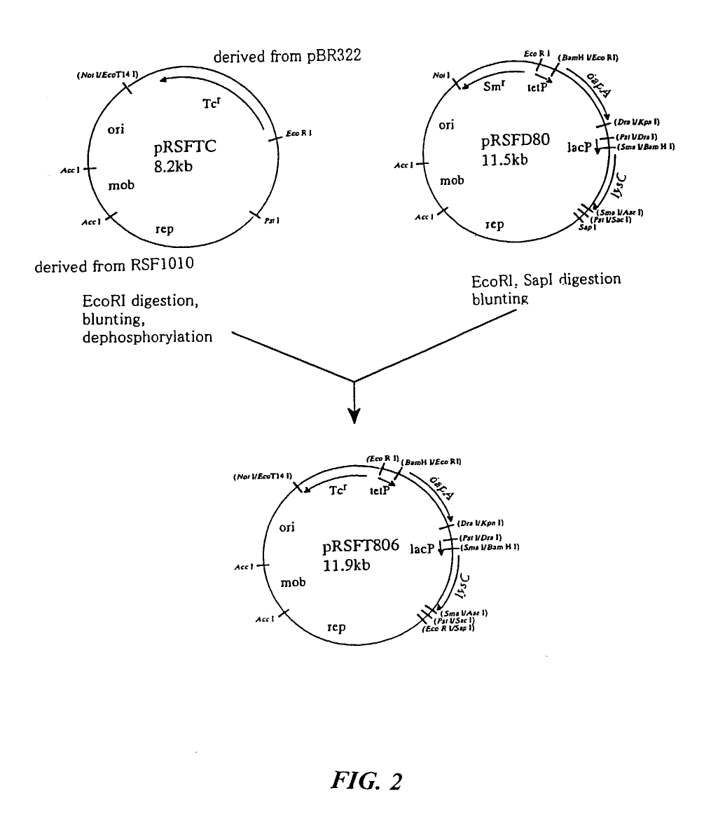

RSFD80 was then digested with the restriction enzymes EcoRI and SapI and the overhangs generated by the restriction enzymes were filled in using T4 DNA polymerase. The resultant digestion was electrophoresed on an agarose gel and the DNA fragment containing the AK and DDPS genes was extracted from the gel using the CONCERT Rapid Gel Extraction System (GIBCO BRL). The plasmid RSFTC was digested with EcoRI, the ends were filled in using T4 DNA polymerase and dephoshorylated using E. coli alkaline phosphatase. The dephosphorylated vector was ligated with the DNA fragment containing the AK and DDPS genes and the ligation reaction was transformed to JM109 (see FIG. 2). Transformants were selected on the agar plate containing 10 mg/l of tetracycline. The resultant plasmid was named as pRSFT806 (see FIG. 2). JM109 that contains pRSFT806 and JM109 that contains pRSFTC were used as donors. A tri-parental conjugal plasmid transfer was performed with JM109 that contains pK2013 as a mobilizer and M. glycogenes ATCC29475 as an acceptor. The plasmids pRSFT806 and PRSFTC were transferred into M. glycogenes respectively and thus M. glycogenes/pRSFTC and M. glycogenes/pRSF806 was obtained selecting on the M1 agar medium containing 10 mg/ml tetracycline.

The M. glycogenes/pRSFTC and M. glycogenes/pRSFT806 were inoculated in M1 medium that contains 3% of calcium carbonate and cultivated by shaking at 30° C. for 36 hours. After cultivation, the cells were removed by centrifugation and L-lysine concentration in the supernatant was measured with the biotech analyzer AS-210 (Sakura Seiki Co., Ltd.). The results were shown in Table 1.

TABLE 1

| Strain | L-lysine Accumulation (g/l) |
|---|---|
| M. glycogones/pRSFTC | not detected |
| M. glcogenes/pRSFT806 | 0.1 |

Incorporation by Reference

Each document, patent application or patent publication cited by or referred to in this disclosure is incorporated by reference in its entirety. Any patent document to which this application claims priority is also incorporated by reference in its entirety. Specifically, priority document JP 300500/1999 filed Oct. 22, 1999 is hereby incorporated by reference.

Obviously, numerous modifications and variations on the present invention are possible in light of the above teachings. It is therefore to be understood that within the scope of the appended claims, the invention may be practiced otherwise than as specifically described herein.

1. A Methylobacillus bacteria comprising an aspartokinase gene wherein the aspartokinase gene is insensitive to feedback inhibition by L-lysine.

2. The Methylobacillus bacteria of claim 1, further comprising a dihydrodipicolinate synthase gene wherein said dihydrodipicolinate synthase gene is insensitive to feedback inhibition by L-lysine.

3. The Methylobacillus bacteria of claim 1, wherein said aspartokinase gene is isolated from Escherichia coli.

4. The Methylobacillus bacteria of claim 2, wherein said a dihydrodipicolinate synthase gene is isolated from Escherichia coli.

5. The Methylobacillus bacteria of claim 2, wherein the aspartokinase gene and the dihydrodipicolinate synthase gene are carried on a plasmid.

6. The Methylobacillus bacteria of claim 2, wherein the aspartokinase gene and the dihydrodipicolinate synthase gene are integrated into the bacterial chromosome.

7. The Methylobacillus bacteria of claim 1 which is Methylobacillus glycogenes.

8. The Methylobacillus bacteria of claim 1 which is Methylobacillus flagelatum.

9. The Methylobacillus bacteria of claim 2, wherein dihydrodipicolinate synthase gene is from Corynebacterium glutamicum.

10. A method of producing L-lysine comprising culturing the microorganism of claim 1 in a media comprising methanol; and collecting the L-lysine produced.

11. A method of producing L-lysine comprising culturing the microorganism of claim 2 in a media comprising methanol; and collecting the L-lysine produced.

12. The method of claim 10, wherein the methanol is in an amount from 0.01 to 30% w/v.

13. The method of claim 11, wherein the methanol is in an amount from 0.01 to 30% w/v.

14. The method of claim 10, wherein said collecting comprises purifying the L-lysine by precipitation.

15. The method of claim 11, wherein said collecting comprises purifying the L-lysine by precipitation.

16. The method of claim 10, wherein said collecting comprises purifying the L-lysine by chromatography.

17. The method of claim 11, wherein said collecting comprises purifying the L-lysine by chromatography.
